(19) United States
(12) Patent Application Publication
(10) Pub. No.: US 2004/0052757 A1
(43) Pub. Date: Mar. 18, 2004

(54) Dermal abrasion facial peel using sodium bicarbonate

(76) Inventor: Gerard J. LeCompte, Pearland, TX (US)

Correspondence Address: Jackie Lee Duke, Attorney at Law, Suite 300, 2500 City West Blvd., Houston, TX, 77042, US

(21) Appl. No.: 10/245,181

(22) Filed: Sep. 17, 2002

(51) Int. Cl. A61K 31/715; A61K 35/78; A61K 33/00

(52) U.S. Cl. 424/74; 424/744; 514/60; 424/717

(57) ABSTRACT

A novel facial peel composition is disclosed for treatment of acne, acne scarring and large skin pores. The facial peel composition is non-surgical, non-laser and contains no acids that can burn or disfigure skin. Its ingredients are all natural and avoids the use of hard non-dissolving abrasives such as aluminum oxide, polymers, plastic or silica. The ingredients include sodium bicarbonate crystals, cornstarch, aloe and water. The sodium bicarbonate crystals are an antibacterial agent that remove acne causing, bacteria laden skin oil. The cornstarch aids in drawing bacteria away from the skin and into suspension for rinsing. The aloe reduces soreness and inflammation to the skin and is a known natural healing agent. A method of preparation of the composition is also disclosed.

### BACKGROUND OF THE INVENTION

1. Field of the Invention

This invention relates to a composition for a dermal abrasion facial peel using sodium bicarbonate as the abrasive agent and the method of its production. It uses proven healing agents and is 100 percent water soluble. Harsh, non-dissolving abrasives such as aluminum oxide or plastic are avoided. Additionally, the facial peel composition contains no acids that can burn or disfigure skin.

It is well known that skin conditions as pimples, blackheads and blemishes are caused by clogging of the skin's pores due to excess oils generated by the body. The excess oils attract dirt, makeup and other foreign bodies to the pores and clog them and thereby cause the aforementioned skin conditions. These skin conditions have been treated for years with various acids, a variety of abrasives or alcohol compounds in an attempt to cleanse the skin of the excess oils.

These typical treatments have all suffered in varying degrees from undesirable and in some cases, painful side effects. The various acid compounds have the problem of scarring or burning the skin, especially the skin of more sensitive users. The abrasives such as aluminum oxide, plastic or silica are not absorbed by the skin but adhere to the skin and if not thoroughly washed from the skin, can themselves contribute to the clogging of pores further exacerbating the skin condition. The alcohol compounds tend to dry the skin excessively leading to other conditions associated with dry skin.

The dermal abrasion facial peel composition of the current invention offers a substantial improvement by providing a composition for exfoliating and cleaning the skin that uses natural ingredients sodium bicarbonate crystals and cornstarch to remove bacteria laden skin oil and a known natural healing agent, aloe vera, to reduce soreness and inflammation of the skin.

2. Description of Related Art

A cosmetic facial preparation containing aloe vera is disclosed in U.S. Pat. No. 4,369,180 to D. M. Mihalovits.

U.S. Pat. No. 4,593,046 to M. Gruber demonstrates a dermatological composition that uses an aloe vera gel in combination with the benzoyl peroxide to ameliorate the skin irritation caused by the peroxide compound.

A topical agent for the treatment of pseudofolliculitis barbae, commonly known as “razor bumps”, containing acetylsalicylic acid, corn starch, isopropyl alcohol and aloe vera is disclosed in U.S. Pat. No. 5,034,221 to S. E. Rosen et al.

U.S. Pat. No. 5,176,918 to J. L. Jones teaches a topical analgesic composition consisting of water, sodium bicarbonate and a transdermal carrier consisting of propylene glycol or equivalent compounds.

A composition and method for the treatment of topical Herpes Simplex 1 infections using an alkali metal carbonate, an alkali metal phosphate, an alkali metal hypochlorite and a topical carrier containing aloe lotion is disclosed in U.S. Pat. No. 5,571,535 to M. Flowers et al.

U.S. Pat. No. 6,063,406 to R. S. Hornack teaches a composition for treating skin disorders consisting of water, calcium chloride, linear long chain phosphate, sodium bicarbonate and polyacrylamide polymer.

### SUMMARY OF THE INVENTION

The present invention provides a novel dermal abrasion facial peel composition for treatment of acne, acne scarring and large skin pores. The facial peel composition is non-surgical, non-laser and contains no acids that can burn or disfigure skin. Its ingredients are all natural and avoids the use of hard non-dissolving abrasives such as aluminum oxide, polymers, plastic or silica.

The ingredients include sodium bicarbonate crystals, cornstarch, aloe and water. The sodium bicarbonate crystals are an antibacterial agent that removes acne causing, bacteria laden skin oil. The cornstarch aids in drawings bacteria away from the skin and into suspension for rinsing. The aloe reduces soreness and inflammation to the skin and is a known natural healing agent. A method of preparation of the composition is also disclosed.

A principal object of the present invention is to provide a facial peel that avoids the use of surgery or laser apparatus to treat acne and acne scarring.

Another object of the present invention is to provide a facial peel composition that uses all natural ingredients that promote healing.

A final object of the present invention is to provide a facial peel composition that avoids the use of harsh acids that can burn or disfigure skin and harsh non-dissolving abrasives.

These with other objects and advantages of the present invention are pointed out with specificness in the claims annexed hereto and form a part of this disclosure. A full and complete understanding of the invention may be had by reference to the accompanying description of the preferred embodiments.

### DESCRIPTION OF THE PRESENTLY PREFERRED EMBODIMENT

The dermal abrasion facial peel composition of the present invention is prepared according to the following method. A quantity of water that will be 25 to 49 percent by weight of the final composition is heated to a temperature sufficient to dissolve 1 to 25 percent by weight of corn starch. This forms a thickened “soup” which is maintained at temperature while 25 to 49 percent by weight of sodium bicarbonate is added. The sodium bicarbonate has a crystalline structure and the sodium bicarbonate crystalline structure preferably comprises the following crystal sizes in the following percentage ranges:

| Size | Minimum Percentage | Maximum Percentage |
|---|---|---|
| 90 micron or less | 0 | 99.6 |
| 150 micron | 0 | 99.6 |
| 250 micron | 0 | 99.2 |

The use of sodium bicarbonate with a range of crystal sizes promotes dissolving of the sodium bicarbonate in the mixture and aids in the cleansing action to be discussed below. This mixture of water, corn starch and sodium bicarbonate is stirred while the final ingredient of 1 to 25 percent by weight of aloe is added. After thorough mixing, the dermal abrasion facial peel composition is allowed to cool to a gel consistency.

The dermal abrasion facial peel composition thus formulated acts in the following manner to cleanse and heal the skin. The skin is cleaned with soapy water and the facial peel composition is then applied using one's finger tips or cotton pads if preferred. The facial peel is worked into the skin for one to five minutes. This allows the sodium bicarbonate crystals to abrade the skin and thereby remove dead skin layers. Additionally, the sodium bicarbonate acts as an antibacterial agent and cleanser to remove acne causing, bacterial laden skin oil. This is done by a process of saponification wherein the oil is absorbed by the sodium bicarbonate crystals which breaks down the oils and are then washed away during rinsing. The rinsing is done with either lukewarm or cold water. The aloe in the dermal abrasion facial peel composition acts as a known natural healing agent to heal the skin and minimize acne scarring.

The composition and method of preparation of my dermal abrasion facial peel will be readily understood from the foregoing description and it will be seen that I have provided a dermal abrasion facial peel composition for treatment of acne, acne scarring and large skin pores. The dermal abrasion facial peel composition is non-surgical, non-laser and contains no acids that can burn or disfigure skin. Its ingredients are all natural and avoids the use of hard non-dissolving abrasives such as aluminum oxide, polymers, plastic or silica. Furthermore, while the invention has been shown and described with respect to certain preferred embodiments, it is obvious that equivalent alterations and modifications will occur to others skilled in the art upon the reading and understanding of the specification. The present invention includes all such equivalent alterations and modifications, and is limited only by the scope of the appended claims.

What is claimed is:

1. A composition for cleaning skin, comprising:
25 to 49 percent by weight of sodium bicarbonate;
25 to 49 percent by weight of water;
1 to 25 percent by weight of corn starch; and,
1 to 25 percent by weight of aloe vera.

2. A composition for cleaning skin according to claim 1, wherein:
the pH of the composition is approximately 8.3.

3. A composition for cleaning skin according to claim 2, wherein:
said sodium bicarbonate has a crystalline structure; and,
said sodium bicarbonate crystalline structure comprises the following crystal sizes in the following percentages:

| Size | Minimum Percentage | Maximum Percentage |
|---|---|---|
| 90 micron or less | 0 | 99.6 |
| 150 micron | 0 | 99.6 |
| 250 micron | 0 | 99.2 |

4. A method of producing a skin cleaning composition, comprising the steps of:
providing 25 to 49 percent by weight of water;
heating said water to a temperature sufficient to dissolve 1 to 25 percent by weight of corn starch;
adding 25 to 49 percent by weight of sodium bicarbonate;
stirring said water, corn starch and sodium bicarbonate composition while adding 1 to 25 percent by weight of aloe vera; and,
allowing said water, corn starch, sodium bicarbonate and aloe vera composition to cool to a gel consistency.

5. A method of producing a skin cleaning composition according to claim 4 wherein:
the composition has a pH in the range of 8.3.
